(19) United States
(12) Patent Application Publication
(10) Pub. No.: US 2013/0165950 A1
(43) Pub. Date: Jun. 27, 2013

(54) SYSTEM AND METHOD TO ACHIEVE RECIPROCATING ROTATIONAL MOTION OF THE DISTAL MEMBER OF A DEVICE BY APPLYING TRANSLATIONAL FORCES

(71) Applicant: BOSTON SCIENTIFIC SCIMED, INC., Maple Grove, MN (US)

(72) Inventor: Carla WIESE, Cincinnati, OH (US)

(73) Assignee: BOSTON SCIENTIFIC SCIMED, INC., Maple Grove, MN (US)

(21) Appl. No.: 13/728,284

(22) Filed: Dec. 27, 2012

Related U.S. Application Data
(60) Provisional application No. 61/580,410, filed on Dec. 27, 2011.

(51) Int. Cl. A61B 17/00

(52) U.S. Cl. CPC A61B 17/00
USPC 606/139

(57) ABSTRACT

A medical device includes an elongate body extending from a proximal portion which, in an operative configuration, remains outside a living body, to a distal end which, in the operative configuration, extends through a body to a position adjacent to a target portion of tissue to be treated and a spool rotatably coupled to the distal portion of the elongate body, the spool including a distal portion coupled to an end effector of the device along with a flexible member extending from the proximal portion of the elongate body, a first portion of the flexible member being wound about the spool in a first direction and a second portion of the flexible member wound about the spool in a second direction opposite the first direction, ends of the first and second portions extending to the proximal portion of the elongate body.

### PRIORITY CLAIM

This application claims the priority to the U.S. Provisional Application Serial No. 61/580,410, entitled “System And Method To Achieve Reciprocating Rotational Motion Of The Distal Member Of A Device By Applying Translational Forces” filed on Dec. 27, 2011. The specification of the above-identified application is incorporated herewith by reference.

### BACKGROUND

Pathologies of the gastro-intestinal (GI) system, the biliary tree, the vascular system and other body lumens and hollow organs are often treated through endoscopic procedures, many of which require active and/or prophylactic hemostasis. Tools for deploying hemostatic clips via endoscopes are often used to stop internal bleeding by clamping together edges of wounds or incisions. These clips grasp tissue surrounding an opening in tissue holding edges of the opening together until natural healing processes have closed the opening Many current clips include a pair of arms which must be in a particular angular orientation to grasp the target tissue edges. Thus, application of the clips requires that they be rotatable by a user. However, it has proven difficult with certain clips to transmit the torque required to rotate clips over the length of the flexible member which connects the clip to the actuator. This difficulty is especially pronounced when the clip device extends along a tortuous path from the actuator to the target tissue.

### SUMMARY OF THE INVENTION

The present invention relates to a medical device, comprising an elongate body extending from a proximal portion which, in an operative configuration, remains outside a living body, to a distal end which, in the operative configuration, extends through a body to a position adjacent to a target portion of tissue to be treated and a spool rotatably coupled to the distal portion of the elongate body, the spool including a distal portion coupled to an end effector of the device along with a flexible member extending from the proximal portion of the elongate body, a first portion of the flexible member being wound about the spool in a first direction and a second portion of the flexible member wound about the spool in a second direction opposite the first direction, ends of the first and second portions extending to the proximal portion of the elongate body.

### BRIEF DESCRIPTION OF THE DRAWINGS

FIG. 1 shows a perspective view of a device according to a first exemplary embodiment of the present invention;

FIG. 2 shows a longitudinal cross-sectional view of a rotation mechanism for rotating a distal portion of the device of FIG. 1;

FIG. 3 shows a longitudinal cross-sectional view of the rotation mechanism of FIG. 1, according to a further embodiment;

FIG. 4 shows a perspective view of a rotation mechanism for rotating a distal portion of a device according to an alternate embodiment of the present invention;

FIG. 5 shows a perspective view of a spool of a rotation mechanism according to another exemplary embodiment of the present invention;

FIG. 6 shows a perspective view of a spool according to yet another exemplary embodiment of the present invention;

FIG. 7 shows a shows a lateral cross-sectional view of the spool of FIG. 6 attached to a distal portion of a device;

FIG. 8 shows a perspective view of a spool according to another exemplary embodiment of the present invention;

FIG. 9 shows a rotation mechanism for rotating a distal portion of a device according to another exemplary embodiment of the present invention;

FIG. 10 shows a perspective view of a spool according to another exemplary embodiment of the present invention;

FIG. 11 shows a side view of the spool of FIG. 10; and

FIG. 12 shows a perspective view of a spool according to yet another exemplary embodiment of the present invention.

### DETAILED DESCRIPTION

The present invention may be further understood with reference to the following description and the appended drawings, wherein like elements are referred to with the same reference numerals. The present invention relates to medical devices requiring the rotation of a distal member and, in particular, relates to a mechanism for rotating the distal member. Exemplary embodiments of the present invention describe a mechanism including a flexible member such as a thread, fiber or wire coiled about a spool and which may be pulled through a shaft of a device to rotate a distal portion of the device in either of a clockwise or a counter clockwise direction. Although the exemplary embodiments specifically describe rotation of a clip assembly at a distal end of a clipping device, it will be understood by those of skill in the art that the mechanism of the present invention may be utilized in any medical device that requires rotation of a distal member located at the end of a long shaft. For example, the mechanism of the present invention may be used for medical devices such as hemostatic clips, biopsy devices, sphincterotomes, retrieval devices, needles, polypectomy snares, endoscopes and radio-frequency ablation devices.

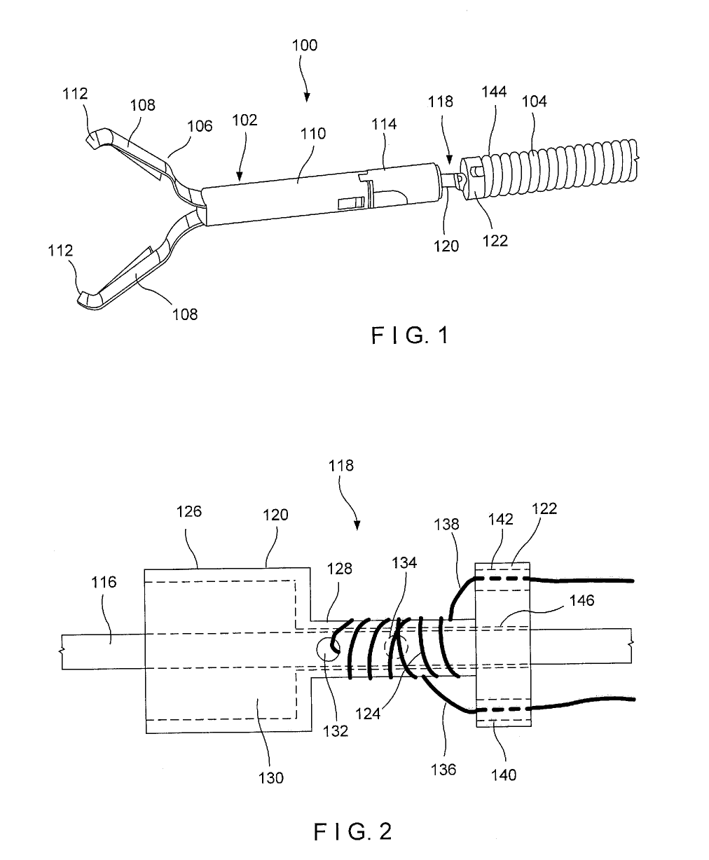

As shown in FIGS. 1-2, a clipping device 100 according to an exemplary embodiment of the present invention comprises a distal assembly 102 connected to a flexible shaft 104 including an actuating handle at a proximal end thereof. Except for the linkage for rotation mechanism, the clip 106 described below, the clipping device 100 may be constructed substantially as disclosed in U.S. patent application Ser. No. 12/853,478, filed Aug. 10, 2010 and entitled “Multifunction Core for Two-Piece Hemostasis Clip,” the entire disclosure of which is expressly incorporated herein by reference. The distal assembly 102 comprises a clip 106 including arms 108 movable within a capsule 110 between an open configuration, in which distal ends 112 of the arms 108 are separated from one another to receive a target tissue therebetween, and a closed configuration, in which the distal ends 112 are moved toward one another to grasp the target tissue therebetween. The capsule 110 is releasably coupled to a bushing 114, from which the capsule 110 may be disengaged during deployment. The clip 106 is movable between the open and closed configurations via a control wire 116 which extends through the shaft 104 from a distal end releasably coupled to the clip 106 to a proximal end connected to the actuating handle. The distal assembly 102 is attached to the shaft 104 via a rotation mechanism 118 including a spool 120, a guide 122 and flexible member 124 which maybe, for example, a filament, fiber, cable, wire, thread or any other suitable flexible member. The spool 120 extends about the control wire 116, connecting the distal assembly 102 to the shaft 104. The flexible member 124 is wound about a portion of the spool 120 with ends of the flexible member 124 guided through the guide 122 to a proximal end of the shaft 104 such as the actuating handle. Thus, as the ends of the flexible member 124 are toggled relative to the shaft 104, a clockwise or counter-clockwise motion of the distal assembly 102 is generated. It will be understood by those of skill in the art that the rotation mechanism 118 is particularly advantageous since the rotation can be controlled and the linear motion of the flexible member 124 is converted to rotation at the distal assembly 102 rather than at a proximal end of the device 100.

The spool 120 includes a distal portion 126, a proximal portion 128 and a lumen 130 extending therethrough to accommodate the control wire 116 therein. In one exemplary embodiment, the spool 120 is substantially tubular. The distal portion 126 is sized and shaped to be engagingly received within the proximal end of the bushing 114 preventing the spool 120 from rotating relative to the distal assembly 102. Alternatively, the distal portion 126 may be fixed within the bushing 114 via, for example, an adhesive, welding or other fixation means preventing relative rotational motion therebetween. The proximal portion 128 has a smaller cross-sectional area (e.g. diameter) than the distal portion 126 and includes first and second holes 132, 134, respectively, extending laterally through opposing portions of the proximal portion 128. The first hole 132 is distal of the second hole 134 and the second hole 134 is spaced a distance distally of the guide 122 selected to accommodate a desired number of windings of the flexible member 124 around the portion of the spool 120 proximal of the second hole 134.

The guide 122 is attached to a distal end 144 of the shaft 104 and may, for example, be substantially disc-shaped. The guide 122 includes first and second holes 140, 142 on opposite sides of the guide 122 along a periphery thereof with the first and second holes 140, 142 open to an exterior of the guide 122. Alternatively, the first and second holes 140, 142 may be completely enclosed. The guide 122 also includes an opening 146 extending therethrough to accommodate the control wire 116 therein. The guide 122 and the proximal portion 128 of the spool 120 are in close proximity to one another but are free to rotate relative to one another.

The flexible member 124 extends through the first and second holes 132, 134 so that a first portion 136 of the flexible member 124 extends to an exterior of the first hole 132 and a second portion 138 of the flexible member 124 extends to an exterior of the second hole 134. The flexible member 124 is sized to pass through the first and second holes 132, 134 across the lumen 120 without interfering with the movement proximally and distally of the control wire 116 extending therethrough to open and close the clip 106. The first and second portions 136, 138 of the flexible member 124 are then wound about the proximal portion 128 in opposite directions. For example, the first portion 136 may be wound about the proximal portion 128 in a clockwise direction while the second portion 138 is wound about the proximal portion 128 counter-clockwise. In some embodiments, the proximal portion 128 may include grooves and/or threads extending thereabout such that the first and second portions 136, 138 extend about the proximal portion 128 within the grooves, aligning the first and second portions 136, 138 with one another. The first portion 136 passes through the first hole 140 of the guide member 122, through the shaft 104 and to a proximal end thereof while the second portion 138 passes through the second hole 142, through the shaft 104 to the proximal end thereof. The first and second portions 136, 138 may extend from the guide 122 through, for example, a single lumen of the shaft 104. Alternatively, the first and second portions 136, 138 may be passed through separate lumens extending through the shaft 104. In another exemplary embodiment, the first and second portions 136, 138 extend through slots extending along a portion of the proximal portion 128 of the spool 120 so that a separate guide 122 is not required.

The ends of the first and second portions 136, 138 are connected to the actuating handle in a manner permitting the end of the first portion 136 to be pulled proximally relative to the device 100 while the end of the second portion 138 moves distally relative to the device 100 and vice versa. As the end of the first portion 136 is pulled proximally, the first portion 136 unwinds from the spool 120 while the second portion 138 winds about the spool 120. As the end of the second portion 138 is pulled proximally, the second portion 138 unwinds from the spool 120 as the first portion 120 winds about the spool 120 rotating the spool 120 and distal assembly 102 including the clip 106 in opposite directions depending on which of the first and second portions 136, 138 is being wound about the spool 120 and which is being unwound. In an exemplary embodiment, a proximal end of the proximal portion 128 abuts against a distal surface of the guide 122 such that the spool 120 bears against the guide 122 as one of the ends of the first and second portions 136, 138 is drawn proximally relative to the device 100. As described above, the spool 120 and the guide 122 are free to rotate relative to one another.

In an exemplary embodiment, the actuating handle at the proximal end of the shaft 104 includes a knob to which proximal ends of the first and second portions 136, 138 of flexible member 124 are attached. The knob is sized and configured such that rotation of the knob in a first direction pulls the first portion 136 of flexible member 124 to rotate the distal assembly 102 clockwise while rotation of the knob in a second direction pulls the second portion 138 to rotate the distal assembly 102 counter-clockwise. It will be understood by those of skill in the art, however, that each of the first and second portions 136, 138 may be controlled together, as described above, or individually via, for example, two knobs. It will also be understood by those of skill in the art that the first and second portions may also be controlled by controllers other than knobs. For example, the controller(s) may include rings, spools, handles, etc., which control the rotation of the first and second portions 136, 138. The actuating handle may also include a mechanism for holding the distal assembly 102 in the desired rotative orientation. The actuating handle may include, for example, a lock, a ratchet mechanism and/or a clamp. In another embodiment, the spool 120 may include a ratchet mechanism and/or a breaking component preventing the flexible member 124 from slipping once one of the first and second portions 136, 138 has been drawn proximally. It will be understood by those of skill in the art, however, that the device 100 may include any of a number of different types of controllers and/or locking/holding mechanisms so long as the first and second portions 136, 138 may be drawn proximally to rotate the distal assembly 102 relative to the shaft 104 and hold the distal assembly 102 at the desired position relative to the shaft 104.

It will be understood by those of skill in the art that many factors will determine how the spool will rotate. For example, rotation may be affected by a diameter of the proximal portion 128 of the spool 120 about which the flexible member 124 is wound and a distance from the holes 132, 134 of the spool 120 to the holes 140, 142 of the guide. Rotation will also be affected by a length of flexible member 124 wound about the proximal portion 128. The longer the portion of the flexible member 124 that is wound about the proximal portion 128, the greater the permitted range of rotation. The positioning of the holes 132, 134 along the proximal portion 128 and a size of the proximal portion 128 may be selected such that he flexible member 124 may be wound therearound to facilitate a rotation of the distal assembly 102 relative to the shaft 104 up to 180° and possibly up to 360°. The spool 120 and the guide 122 may be formed of, for example, any suitable biocompatible plastic or metal. Portions of the spool 120 and/or guide 122 may have different surface textures to provide a smooth passage for the flexible member 124 or to generate additional friction to enhance an amount of torque applied to the spool 120. The spool 120 and the guide 122 may be manufactured via, for example, stamping, machining or injection molding.

According to an alternate embodiment, the flexible member 124 may include a tacky section along a portion of the flexible member 124 which will be wound around the proximal portion 128 of the spool 120 such that the spool 120 does not require holes 132, 134. The tackiness of the flexible member provides the friction required to rotate the spool 120 as it is wound and unwound. The flexible member 124 may be formed of a tacky material and/or have a tacky material impregnated into it or attached to it. The spool 120 may be made of any of a variety of materials which may increase the friction between the flexible member 124 and the spool 120 and/or an outer surface of the spool 120 or of a portion of the spool 124 over which the flexible member is to be wound may be formed or treated to increase the frictional coupling of the flexible member and the outer surface of the spool 120 as would be understood by those skilled in the art. For example, the spool 120 may also be formed of a tacky material and/or have a tacky material impregnated into it or attached to it. The friction between the flexible member 124 and the spool 120, however, may be increased in any number of ways. For example, the spool 120 and/or flexible member 124 may be treated with a coating, the spool 120 may have different surface areas and/or include a surface roughness, and/or a a tension of the flexible member 124 wound about the spool 120 may be increased.

According to another embodiment, the flexible member 124 may be wound about the proximal portion 128 of the spool 120 in a single direction such that pulling an end of the flexible member 124 rotates the distal assembly 102. The flexible member 124 may be wound about the proximal portion 128 to permit multiple spins of the spool 120 such that the clip 106 may rotate up to 360°. Thus, the clip 106 may be rotated to any desired orientation relative to the target tissue by pulling on a single end of the flexible member 124.

It will be understood by those of skill in the art that although the exemplary embodiment specifically describes one flexible member 124, the device 100 may include two flexible members 124, each of which may be attached to the spool 120 via, for example, an adhesive, knots, fasteners, etc., and wound about the proximal portion 128 in opposite directions. Thus, it will also be understood by those of skill in the art that although the device 100 is specifically described as including holes 132, 134 through which the flexible member 124 may be inserted, the flexible member 124 may be attached to the spool 120 in any of a variety of ways so long as the flexible member(s) 124 may be wound thereabout.

As shown in FIGS. 1-2 and described above, portions of the spool 120, the guide 122 and the flexible member 124 extend between a proximal end of the bushing 114 and the distal end 144 of the shaft 104. In these cases, a protective sheath may extend over the rotation mechanism 118 to protect the rotation mechanism 118 as the device 100 is inserted into the body. In another embodiment, however, the proximal portion 128 of the spool 120 and the guide 122 may be positioned within the shaft 104 such that the rotation mechanism is not exposed to an exterior of the device 100. In yet another embodiment, the distal assembly 102 may be connected to the shaft 104 via a spinlock, which would permit the distal assembly 102 to be rotated relative to the shaft while preventing the distal assembly 102 from being separated from the shaft 104. It will be understood by those of skill in the art that this embodiment would be particularly useful for medical devices in which the distal assembly 102 is not required to be deployed from a proximal portion of the device 100.

According to an exemplary technique in accord with the present invention, the distal assembly 102 of the device 100 is inserted to a target area within a body (e.g., through the working channel of an endoscope inserted into a body lumen via a naturally occurring bodily orifice) until the clip 106 is positioned proximate target tissue to be clipped. The control wire 116 is then moved distally relative to the shaft 104 to allow the clip arms 108 to spread apart (e.g., under their natural bias) into the open configuration. The user observes the clip (e.g., via the vision system of an endoscope) and determines whether the clip arms 108 need to be rotated to properly clip the target tissue. If the clip is not in the desired rotational orientation relative to the target tissue to be clipped, the user operates an actuator to rotate the distal assembly 102 relative to the shaft 104 to the desired rotational orientation relative to the target tissue via the rotation mechanism 118 as described above. Either of the ends of the first and second portions 136, 138 may be moved proximally relative to the shaft. As described above, pulling the end of one of the first and second portions 136, 138 rotates the distal assembly 102 either clockwise or counter-clockwise relative to the shaft 104. Once the clip 102 has been placed in the desired rotational orientation relative to the target tissue, the clip 102 is advanced distally until the target tissue is positioned between the distal ends 112 of the clip arms 108 and the control wire 116 is drawn proximally relative to the shaft 104 to move the clip arms 108 to the closed configuration, gripping the target tissue between the distal ends 112. When the user determines that the desired portion of tissue is properly grasped between the arms 108, the control wire 116 is drawn farther proximally sever the link between the control wire 116 and the clip 106, locking the clip arms 108 in the closed configuration over the tissue separating the capsule 110 from the bushing 114 so that the clip 106 is completely separated from the proximal portion of the device 100 with the clip 106 locked on the target tissue.

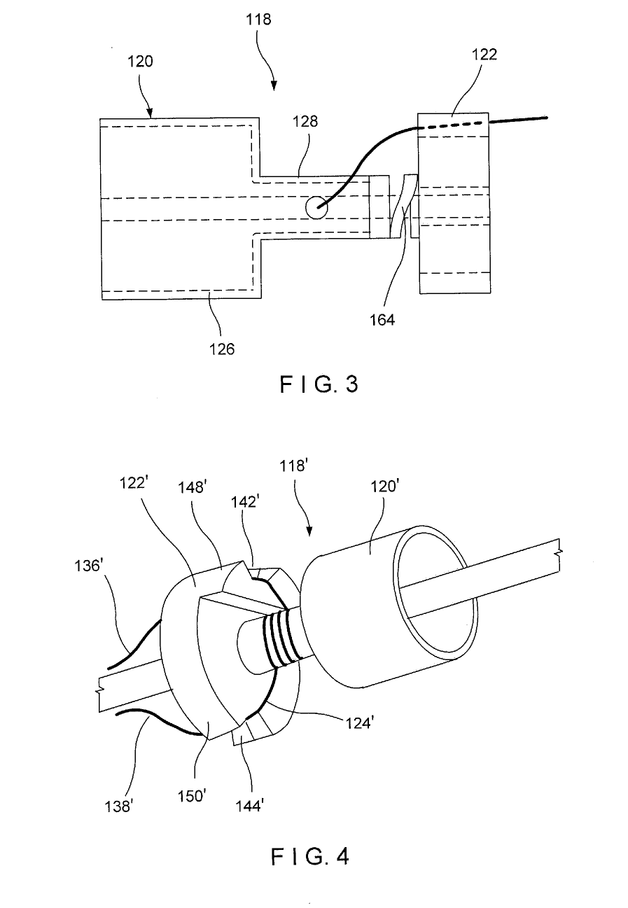

According to an alternate embodiment of the present invention, as shown in FIG. 3, the rotation mechanism 118 may further comprise a torsional spring 164 positioned between the spool 120 and the guide 122, permitting the spool 120 and the distal assembly 102 to be rotated relative to the shaft 104 in a first direction with the spring rotating the device back to an initial orientation when the actuator is released by a user as would be understood by those skilled in the art. In this embodiment, the flexible member 124 does not require first and second portions 136, 138 wound about the spool 120 in opposite directions. Rather, a single portion of flexible member 124 may be wound in a single direction about the spool 120 over a length sufficient to rotate the clip 106 as far from the initial orientation (e.g., 180°)as required to achieve any desired rotational orientation of the clip 106. Pulling the flexible member 124 proximally relative to the shaft 104 moves the distal assembly 102 in the first direction. Once the flexible member 124 is released, however, the distal assembly 102 rotates in the second direction to revert to the original orientation.

In another embodiment, the spring 164 may be preloaded (e.g., partially twisted prior to use) such that release of an end of the flexible member 124 causes the distal assembly 102 to rotate relative to the shaft 104 in a first direction. Once released, the end of the flexible member 124 may be drawn proximally relative to the shaft 104 to rotate the distal assembly 102 in a second direction to provide bi-directional control with a single flexible member 124.

A rotation mechanism 118′, as shown in FIG. 4, is substantially similarly to the rotation mechanism 118, described above, comprising a spool 120′, a guide 122′ and a flexible member 124′, except that the guide 122′, although similar to the guide 122 in other respects, includes a first portion 148′, through which a first hole 140′ extends for receiving a flexible member 124′ has a greater width (i.e., distance between a proximal and a distal end thereof) than a second portion 150′ of the guide 122′ through which a second hole 142′ extends for receiving the flexible member 124′. The difference in widths between the first and second portions 148′, 150′ permits a first portion 136′ of the flexible member 124′ passing through the first hole 140′ to be guided away from a spool 120′ at a predefined distance from a second portion 138′ of the flexible member 124′passing through the second hole 142′ such that the first and second portions 136′, 138′ have more room to wind and unwind about the spool 120′, thereby preventing interference therebetween.

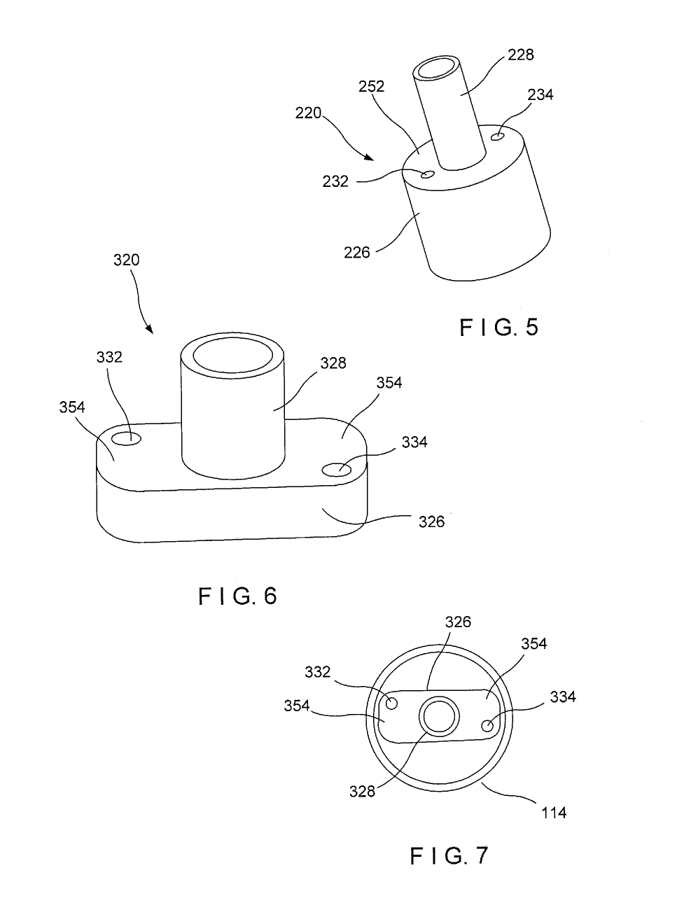

As shown in FIG. 5, a spool 220, according to another exemplary embodiment of the present invention, is substantially similar to the spool 120, described above and may be utilized in place of the spool 120 in the rotation mechanism 118. The spool 220 similarly includes a distal portion 226 sized, shaped and configured to be engagingly received within the bushing 114 and a proximal portion 228 having a smaller cross-sectional area than the distal portion 226. However, rather than holes extending through the proximal portion 228, however, first and second holes 232, 234 extend longitudinally through a proximal surface 252 along central axes substantially perpendicular to a longitudinal axis of a lumen 230 of the spool 220, through which the control wire 116 extends, on opposing sides thereof. Thus, the flexible member 124 may be threaded through the first and second holes 232, 234 such that the proximal and distal portions 136, 138 extend proximally therefrom to be wound about the proximal portion 228.

As shown in FIGS. 6-7, a spool 320, according to another exemplary embodiment of the present invention is substantially similar to the spool 220, described above, including a distal portion 326 and a proximal portion 328. The distal portion 326, however, is elongated in a lateral direction to form a pair of wings 354 extending on opposing sides of the proximal portion 328. These wings 354 are coupled to the distal assembly 102 and a first hole 332 extends longitudinally through a first one of the wings 354 while a second hole 334 extends through a second one of the wings 354. As shown in FIG. 7, the wings 354 may be received within the bushing 114 of the distal assembly 102 and affixed thereto via, for example, welding or an adhesive. It will be understood by those of skill in the art that the wings 354 may have any of a variety of shapes and orientations relative to a longitudinal axis of the spool 320. The flexible member 124 is inserted through the first and second holes 332, 334 similarly to the spool 220 and operates in a manner that is otherwise similar to the spools described above.

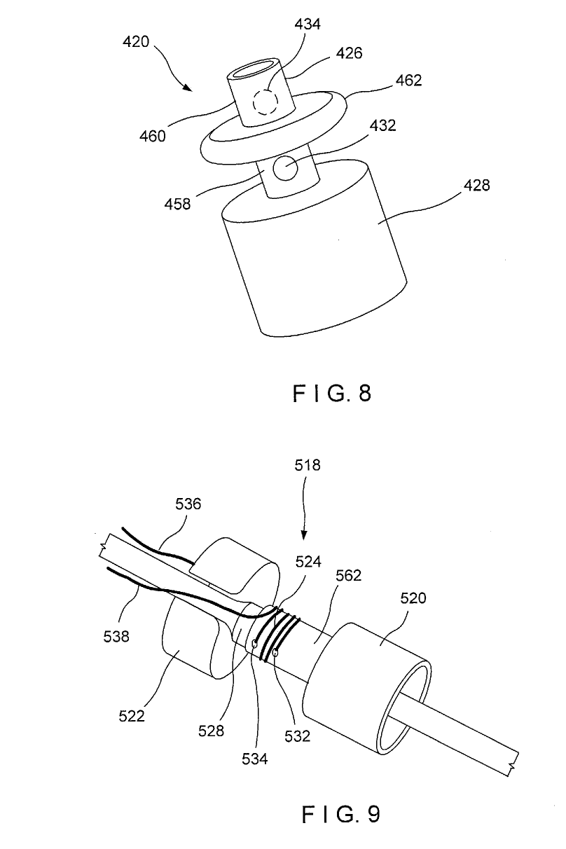

As shown in FIG. 8, a spool 420 according to another exemplary embodiment is substantially similar to the spool 120, described above, including a distal portion 426 for engaging the distal assembly 102 and a proximal portion 428 about which the flexible member 124 may be wound. The proximal portion 428, however, is divided into a first section 458 and a second section 460 separated from one another along a length of the proximal portion 428 via a laterally extending shoulder 462 extending radially outward from the proximal portion 428 in a substantially disc-like shape. A first hole 432 extends laterally through a wall of the first section 458 while the second hole 434 extends laterally through the second section 460, along a portion of the wall opposing the first hole 432 but offset longitudinally therefrom. The flexible member 124 may be threaded through the first and second holes 432, 434 such that the first portion 136 of flexible member 124 extends out of the first hole 432 to be wound about the first section and the second portion 138 of flexible member 124 extends out of the second hole 434 to be wound about the second section 460. The separated sections 458, 460 prevents the first and second portions 136, 138 of flexible member 124 from translating over the other.

As shown in FIG. 9, a rotating mechanism 518 according to a further exemplary embodiment comprises a spool 520, a guide 522 and a flexible member 524 substantially similar to the spool 120, guide 122 and flexible member 124 of the rotating mechanism 118 described above. The rotating mechanism 518, however, further comprises a cuff 562 slidable over a proximal portion 528 of the spool 520 via, for example, an interference fit, welding, or other fixation mechanism. In this embodiment, the spool 520 includes no holes therethrough to receive the flexible member 524. Rather, the cuff 562 includes first and second holes 532, 534, respectively, extending laterally through a wall thereof. The first and second holes 532, 534 are separated from one another along a length of the cuff 562, about a circumference of the cuff 562 and/or positioned along the same side of the cuff 562. The flexible member 524 is inserted through the first and second holes 532, 534 such that a first portion 536 of flexible member 524 extends to an exterior of the cuff 562 from the first hole 532 and a second portion 538 of flexible member extends to an exterior of the cuff 562 via the second hole 534. The cuff 562 may then be slid over the proximal portion 528 of the spool 520 such that a portion of the flexible member 524 within the cuff 562 extends about the proximal portion 528 and/or between the proximal portion 528 and the cuff 562, securing the flexible member 524 therebetween. The first and second portions 536, 538 of flexible member 524 are then wound about the cuff 562 in opposite directions and guided through the holes of the guide 522, as described above in regard to the device 100.

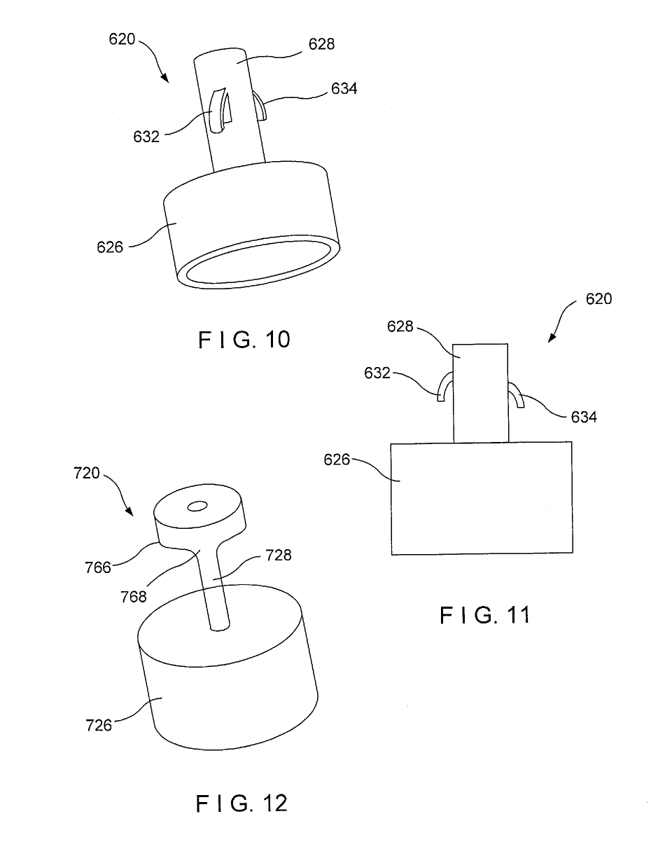

As shown in FIGS. 10-11, a spool 620 according to a further exemplary embodiment is substantially similar to the spool 120 described above in regard to the device 100, comprising a distal portion 626 for attachment to the distal assembly 102 and a proximal portion 628 about which the flexible member 124 may be wound. Rather than holes 128, 134 through which the flexible member 124 may be inserted, however, the spool 620 includes first and second hooks 632, 634 extending from a portion of the proximal portion 628 toward a distal end thereof such that the flexible member 124 may be hooked therein and wound about the proximal portion 628 proximally of the lances 632, 634. The first and second hook 632, 634 are positioned along substantially opposing portions of the proximal portion 628. Although the exemplary embodiment specifically describes two hooks 632, 634, it will be understood by those of skill in the art that the spool 620 may include one hook for hooking the flexible member 124.

As shown in FIG. 12, a spool 720 according to a further exemplary embodiment may be substantially similar to any of the spools 120-620 described above, comprising a distal portion 726 for attachment to the distal assembly 102 and a proximal portion 728 about which the flexible member 124 may be wound. The spool 720, however, further comprises a flange 766 at a proximal end 768 of the proximal portion 728. The flange 766 extends radially outward from the proximal end 768 such that a flexible member 124 wound about the proximal portion 728 is prevented from sliding proximally past the proximal end 768.

It will be apparent to those skilled in the art that various modifications and variations can be made in the structure and methodology of the present invention, without departing from the spirit or scope of the invention. Thus, it is intended that the present invention cover the modifications and variations of this invention provided that they come within the scope of the appended claims and their equivalents.

What is claimed is:

1. A medical device, comprising:
an elongate body extending from a proximal portion which, in an operative configuration, remains outside a living body, to a distal end which, in the operative configuration, extends through a body to a position adjacent to a target portion of tissue to be treated;
a spool rotatably coupled to the distal portion of the elongate body, the spool including a distal portion coupled to an end effector of the device; and
a flexible member extending from the proximal portion of the elongate body, a first portion of the flexible member being wound about the spool in a first direction and a second portion of the flexible member wound about the spool in a second direction opposite the first direction, ends of the first and second portions extending to the proximal portion of the elongate body.

2. The device of claim 1, wherein the spool includes first and second holes through which the flexible member extends.

3. The device of claim 1, wherein the spool includes grooves extending thereabout to guide the flexible member about the spool.

4. The device of claim 2, wherein the flexible member passes through first and second holes extending through the spool.

5. The device of claim 4, further comprising:
a control member extending along a longitudinal axis of the device through a first lumen within the elongate body, a second lumen within the spool and a third lumen extending through the spool, the first and second holes extending through the winding portion of the spool transverse to and opening into the third lumen.

6. The device of claim 5, wherein the first hole is separated from the second hole along the longitudinal axis of the device.

7. The device of claim 2, wherein the first and second holes extend through the distal portion of the spool substantially parallel to the longitudinal axis.

8. The device of claim 2, wherein the distal portion forms two arms extending laterally therefrom transverse to the longitudinal axis, the first hole extending through a first one of the arms and a second hole extending through a second one of the arms, the first and second holes extending substantially parallel to the longitudinal axis.

9. The device of claim 1, wherein a portion of the guide through which the first opening extends is wider in a direction substantially parallel to the longitudinal axis than a portion of the spool through which the second opening extends.

10. The device of claim 1, wherein the first and second openings of the spool are formed as channels open through a radially outer surface of the guide.

11. The device of claim 1, wherein the first and second openings are formed as holes open only through the proximal and distal surfaces of the spool.

12. The device of claim 1, further comprising:
a cuff slidably received over the winding portion of the spool, the cuff including third and fourth holes extending with the flexible member extending therethrough.

13. The device of claim 1, wherein at least a portion of one of the flexible member and the winding portion of the spool is treated to enhance a frictional coupling therebetween.

14. The device of claim 13, wherein a portion of the flexible member wound about the spool includes a tacky material increasing a frictional engagement between the spool and the flexible member.

15. The device of claim 4, wherein the end effector is hemostatic clip coupled to the spool so that, movement of the flexible member winding and unwinding the flexible member over the winding portion of the spool rotates the spool and the clip relative to the elongate member.

16. The device of claim 15, wherein the clip is housed in a capsule releasably coupled to the spool via a bushing with the control member extending through the bushing and the capsule to couple to the clip via a connection designed to fail when a tension applied to the control member exceeds a predetermined threshold level.

17. The device of claim 1, wherein the spool includes a flange extending radially outward from a distal end thereof to prevent the flexible member from being disengaged therefrom.

18. The device of claim 1, wherein the spool includes a hook extending therefrom to hook the flexible member thereto.

19. A method for treating tissue, comprising:
inserting to a target area within a living body, a flexible elongate body of a device including a distal member coupled to the elongate body via a rotation mechanism, the rotation mechanism including a spool rotatably coupled to a distal end of the elongate body and a flexible member wound about the spool; and
drawing a proximal end of the flexible member proximally to partially unwind the flexible member from the spool, rotating the spool and an end effector non-rotatably coupled thereto relative to the elongate body.

20. The method of claim 19, further comprising:
drawing a first end of the flexible member proximally to unwind a first portion wound about the spool in a first direction thereby rotating the spool relative to the elongate body in a first direction and drawing a second end of the flexible member proximally, the second portion being wound about the spool in a second direction, to rotate the spool relative to the elongate body in a second direction.
